(19) United States
(12) Patent Application Publication
(10) Pub. No.: US 2008/0060653 A1
(43) Pub. Date: Mar. 13, 2008

(54) Mask System with Improved Sealing Properties for Administering Nasal Positive Airway Pressure Therapy

(76) Inventors: Michael David Hallett, Sydney (AU); Michael Kassipillai Gunaratnam, Marsfield (AU)

Correspondence Address: Dr. Michael Hallett, Unit 803 / 8 Distillery Drive, Pyrmont, 2009, omitted

(21) Appl. No.: 11/850,686

(22) Filed: Sep. 6, 2007

(30) Foreign Application Priority Data
Sep. 11, 2006 (AU) 2006904949

(51) Int. Cl. A61M 16/06

(52) U.S. Cl. 128/206.24

(57) ABSTRACT

An improved mask sealing apparatus is described for use in nasal positive pressure therapies such as nasal CPAP (continuous positive airway pressure) and nasal ventilation for treatment of such disorders as sleep apnea, ventilatory insufficiency and complex sleep apnea. The device most importantly provides a means for formation of 2 sealing zones when fitted to a user's face. The first is formed at or within the nares of a user, while a second sealing zone is formed around a user's nose. The cushion so formed may be constructed as a thick walled profile or a thin walled profile using a materials of appropriate durometer. Various configurations of the nares and peri-nasal sealing components are further described.

### CROSS REFERENCE TO RELATED APPLICATIONS

This patent is a full specification based on Australian provisional patent application number 2006904949

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND TO THE INVENTION

Positive pressure therapies include nasal continuous positive airway pressure (CPAP) as used in the treatment of obstructive sleep apnea (OSA) and nasal intermittent positive pressure ventilation for breathing support in chronic restrictive and obstructive lung diseases and associated acute exacerbations. These therapies frequently employ a nasal mask connected by a tube or flexible conduit to a source of pressurized breathable gas such as a positive pressure air flow generator. The mask system typically consists of a flexible mask cushion, which also acts as a gasket to prevent loss of gas pressure from inside the mask, attached to a mask frame or manifold attached to the users head via a head strap or head gear. The mask assembly usually covers the nose but can also cover both the nose and mouth to circumvent mouth leaks and breathing to atmosphere. Examples of such mask and cushion devices are well established in the prior art and are described in several patents examples of which include U.S. Pat. Nos. 6,959,710, 6,871,649, 5,746,201 and 5,724,965. Alternatively, the device may be designed to sit superficially on or be partially inserted into a user's nares. These are also described extensively in the prior art, for example as defined in U.S. Pat. Nos. 7,000,613, 6,119,694, 5,042,478 and 4,782,832. These devices however often remain suboptimal by virtue of propensity to leak depending on the user's position and applied therapy pressure.

The invention described herein takes advantage of the benefits of both a nares seal and peri-nasal seal in a single convenient design to provide a double gasket or multiple stage seals. In this way the seal may be optimized.

There are numerous benefits from reducing undesired leaks from a mask system for use with positive pressure therapies, and these include:

1) The source of pressurised gas, such as provided by a fan blower, and oxygen (if added) is used most efficiently.

2) Leak related side effects, such as noise and blow past, which are a major source of patient discomfort and hence non-compliance, are reduced.

3) ‘Rise times’ i.e. the time intervals over which maximum treatment pressure is delivered may be reduced, which has importance in the treatment of more severe respiratory disorders e.g. chronic or acute exacerbations of obstructive pulmonary disease.

4. Reducing leak optimizes external heating and humidification when used.

### SUMMARY OF THE INVENTION

The invention takes advantage of multiple-stage seals. The goal of this sealing arrangement is to provide multiple sealing zones:

1. A primary sealing zone in the vicinity of the nares.

2. A secondary sealing zone peripheral to and around the nose.

3. Optionally, further sealing zone(s) beyond the periphery of the secondary sealing zone.

There features are shown in FIG. 1 where 1 represents the primary sealing zone at or within the nares and 2 represents the secondary sealing zone around the nose and over the nasal bridge.

The secondary sealing zones peripheral to the nose comprise a complex and varying topology, which is notoriously difficult to seal. This is due to the fact that some areas lie relatively perpendicular to the direction of sealing force applied by the headgear e.g. the cheeks and below the nose, and others lie relatively tangential e.g. the sides of the nasal bridge. Areas that are relatively tangential require higher magnitudes of sealing force, leading to discomfort and increasing the risk of patient non-compliance, or alternatively require the added complexity of mechanisms to increase perpendicular sealing force components such as ‘pinching’ devices at the nasal bridge.

Leaks in these hard-to-seal zones, such as the sides of the nasal bridge, are likely to introduce side-effects such as leaks into the eyes, which can cause acute irritation and discomfort and in extreme cases may lead to long-term complications.

Additional embodiments are provided to enhance sealing in the area of the nasal bridge including a sculptured fit in this area and providing a thin flexible membrane to provide a stretch seal.

The method of exhalation may include passive continuous flow from an external vent to atmosphere or exhalation valve.

### BRIEF DESCRIPTION OF THE DRAWINGS

FIG. 1 (Title Page): In cases where the naring seals (primary sealing zones) fail to seat securely and air leaks into the surrounding chamber (secondary sealing zone) the seal provided around the nose will mitigate further leak providing that seal is integral or more integral than provided by the primary zone. In that case, secondary zone pressure will be close to the delivered pressure. If the naring seals are well positioned, providing no leaks, then the pressure in the secondary zone will be low or close to atmospheric depending on the sealing between the secondary zone and atmosphere.

FIG. 2: The rim of the nares requires a face seal approach where a continuous sealing force approach is applied in the direction of the arrow

FIG. 3: Sealing inside the nostril requires the application of radial sealing force in the general directions of the arrow cluster

FIG. 4: The basic concept of a multi-stage seal as applied to a mask system. Arrows show air flow direction depending on inhalation or exhalation. Venting system not shown in mask frame

FIG. 5: Illustrates the ‘patient side’ of a cushion concept

FIG. 6: Illustrates the ‘mask side’ of a cushion concept

FIG. 7: The mask around the bridge may be sculptured to provide a more close profile with the nasal bridge area

FIG. 8: The bridge area may be fitted with a stretchable elastomeric membrane that stretches over the nasal bridge to further enhance an air tight seal

FIG. 9: Shows orientation of the nares sealing component with gas holes directed upward

FIG. 10: Shows orientation of the nares sealing component with gas holes directed upward and outward as a possible orientation to ease molding

FIGS. 11A and 11B: A further embodiment where the nares seal may be created in the cushion and the tool core later refilled

FIGS. 12A and 12B: A further embodiment where a nares seal pocket is first created in a cushion and the seals are placed in the pocket to complete the cushion. The pocket is shown such that the mold core is removed from the lower part of the cushion.

FIGS. 13A and 13B: A further embodiment where the nasal protrusions may be contoured to have a low profile or exaggerated profile depending on a user's facial and nares profile.

FIG. 14: An embodiment where an elastomeric thin walled cushion with shaded sections indicating optional features such as a forward nasal section and flanges for attaching the cushion to a mask frame

FIG. 15: A further embodiment shown in FIG. 11 where the primary seals are of a bellows form.

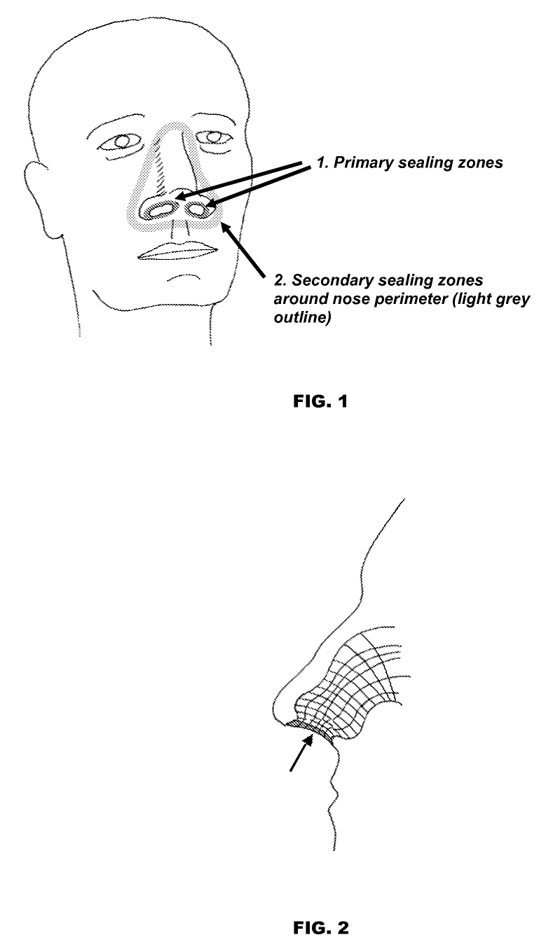

### DETAILED DESCRIPTION OF THE INVENTION

The principal of operation may be described by equation 1 where the total resistance to airflow (R_{T}) cmH20/l/min between the interior of a mask and the atmosphere is given by

R_{T}=R₁+R₂ (1)

In such as system R_{T} should be high and in this case the resistance will be provided by seals or flow resistances to leak or non lung directed flow at primary sealing zone at the nares (R1) and secondary zone on the user's face (R2).

The primary sealing zone at the nares will be considered first.

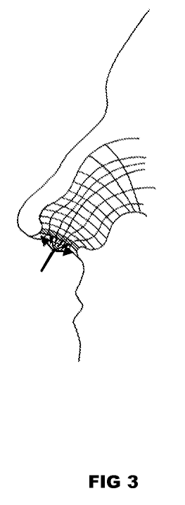

Sectioned views of a nose are shown in FIGS. 2 and 3 and indicate that there are two key sealable zones, each of which calls for a different approach to seal.

It can be seen that the interior of the nasal passage possesses a complex topology, while the entry to the nasal passage possesses substantially an ‘elliptic donut’ topology.

Sealing at the rim of the nares is shown in FIG. 2, and requires a seal whose ultimate reaction is transmitted to the headgear.

The advantages of this approach are:

1. The mask is relatively easy to don and doff, and does not require careful insertion of the sealing portion.

2. Rim sealing cushions have less dramatic protuberances and may permit more complex cushion structures to be tooled/molded.

3. The seal is potentially less intrusive, and applies less direct pressure within the nares to the wearer, aiding comfort.

However, to establish and maintain a seal, force must be maintained with a component perpendicular to the nares. This requires the headgear to provide forces in an additional direction to those required with masks as described in the prior art. Hence, increased headgear complexity may be required in order to prevent a situation where small head/neck movements permit seal force fluctuations and consequent leaks.

Sealing inside of the nostril wall (within the nostril cavity) requires the application of a radial/outwards sealing force as shown in FIG. 3. This may be provided by for example a flexible, elastic structure which distends outwards under the application of treatment pressure, or, by the insertion of a structure having dimensions sufficiently large relative to the nose, so as to create an ‘interference fit’.

In the latter case, the structure may either be relatively elastic, and deformable to comply with the shape of the nostrils, or it may be relatively rigid, and rely upon the elasticity of the nose to comply with it.

To amplify the sealing effect at or in a user's nares the mask is provided with a further sealing area around the nose periphery or perimeter thus producing a multi stage sealing or gasket arrangement. The periphery would typically include an area at the nasal bridge and an area around the periphery of the nose. The periphery will generally include the upper lip below the nostrils, and area between the nasal contour up to and including the cheek bones and sides and top of the nasal bridge.

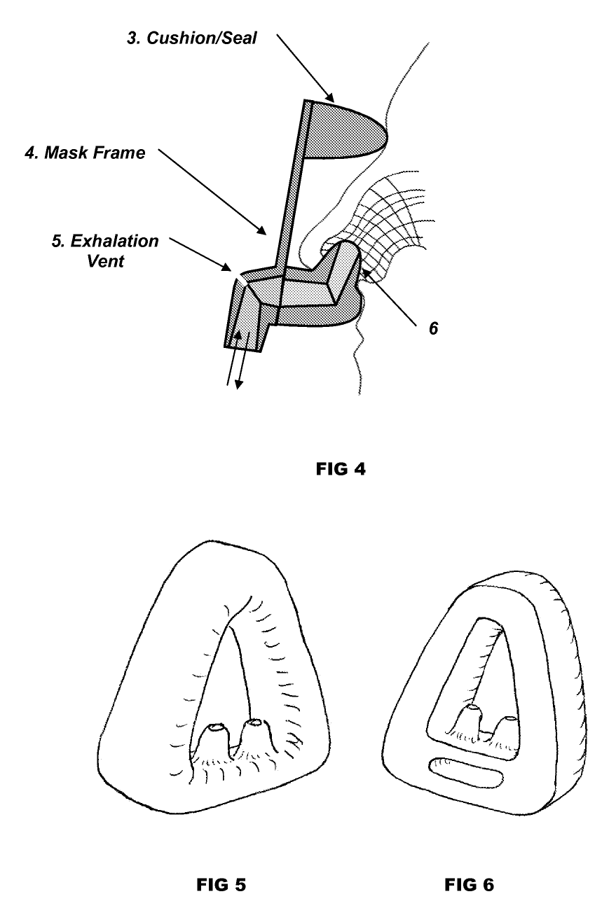

A stylized concept of a multi-stage seal as applied to a mask system is illustrated in FIG. 4. In this case the nares seal is provided by an interference fit by being inserted into the nares at 6, but may also sit on the surface of the nares as described above and as shown in FIG. 2. Secondary sealing is providing by cushion 3, venting is provided here by a fixed vent at 5 in mask frame 4.

FIGS. 5 and 6 show a view of one possible mask cushion configuration. In this case the naring seals and surrounding nasal cushion are molded from a single piece of elastomeric material. FIG. 5 represents the front view of the cushion, or the surface which is in contact with the user's face and nares. FIG. 6 represents the rear view, or surface which is in contact with the mask frame assemble for providing attachment points to a headgear arrangement.

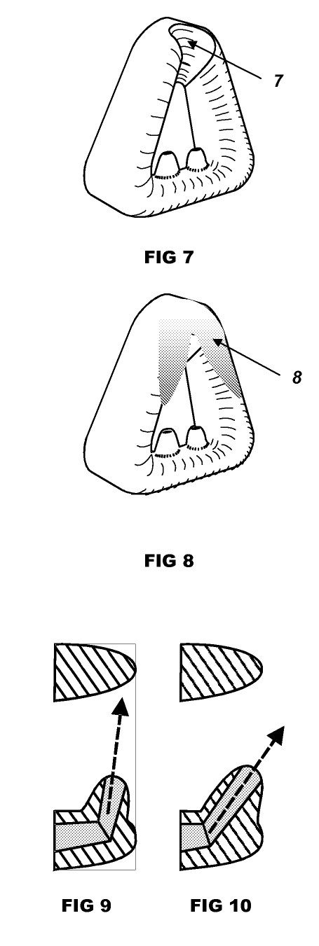

In can be appreciated by the preceding discussion that the secondary sealing area may be provided exclusively by the cushion body. As shown in FIG. 7 the secondary sealing area may be further enhanced by providing a sculptured area at 7 to conform more closely with the nasal bridge area.

In a further embodiment as shown in FIG. 8 a flexible membrane may be attached, by bonding for example, to the cushion at the region where the cushion contacts the nasal bridge area and angled structures to the surface of the face. This flexible membrane at 8 acts to stretch over the protruding nasal bridge to enable an enhanced seal over that provided by the supporting cushion structure. Optionally this stretch membrane may be extended around the perimeter of the cushion.

FIG. 9 illustrates a manufacturing tooling point of interest with the cushion structure as implemented in its simplest form. The mold core used to form part of the nasal channel can be obstructed by the top of the mask cushion. FIG. 10 shows that angling the nasal channel may resolves a potential tooling issue. The direction of the mold core withdrawal in indicated by arrows.

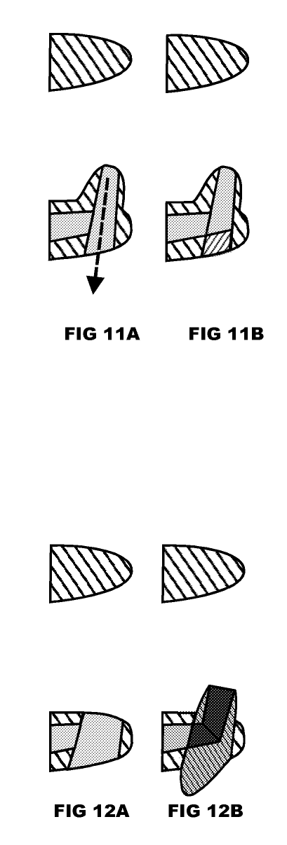

A further embodiment is shown in FIG. 11A and is designed to address possible manufacturing issues discussed above. Here construction of the nasal channel during molding is from below, which requires a subsequent operation to plug the lower hole as in FIG. 11B. Alternatively this hole could be utilized for a function such as pressure measurement. Direction of the mold core withdrawal in indicated by the arrow.

In yet a further embodiment shown in FIG. 12A shows a cushion having an orifice formed into which nasal seal insert(s) may be installed as shown in FIG. 12B. The insert may be custom molded by rapid prototyping techniques to suit an individual user, or mass produced according to a standardized range of anthropometric fits. The insert may feature lower projections to assist the wearer in manipulating the seal into the nares. These projections may also be shaped to mitigate inadvertent aspiration of the seal. The seal may be removably installed or permanently glued or similarly placed into position.

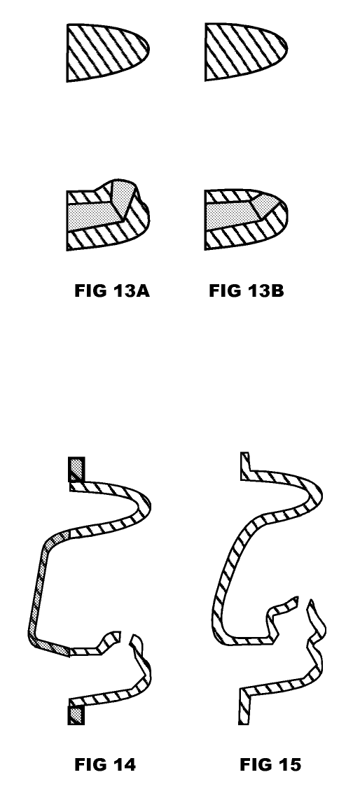

FIGS. 13A and 13B show embodiments with discrete or no primary protruding seals which would seal on the surface of the nares at the entry to the nasal cavity. This would be manufactured to suit a wider set of facial features and user preferences, and would be designed to enable insertion into the nares or to be seated on the surface of the nares or partly within the nares.

In yet a further embodiment, FIG. 14 shows an elastomeric thin walled cushion/seal with shaded sections indicating optional features such as a sealed forward nasal section and flanges for attaching to a mask frame. In this case the nares sealing component is molded into the cushion wall.

FIG. 15 shows a variation of FIG. 14 where the primary nasal seals are of a bellows form. The bellows may include multiple bellow folds which would also extend when a gas pressure is applied within the cushion and attached frame and thereby enhancing the sealing pressures at the nares surface.

It should be noted that where cushion/seal structures incorporate undercuts or other features that preclude molding by mass-production tools, custom or rapid prototype techniques such as elastic (e.g. silicone) molds and/or rotational or dip molding for thin walled structures may be applied.

Heavy walled cushion/seals e.g. FIGS. 4 to 13 would preferably be made from a low durometer elastomer such as silicone or a thermoplastic elastomer (TPE) preferably of durometer 0 to 40 Shore A. In order to overcome the inherent contact ‘stickiness’ of low durometer elastomers, the component may be coated, dipped or sprayed with a less sticky material, typically an elastomer of higher durometer.

While the invention has been described with reference to a range of embodiments as described above, it will occur to those skilled in the art that various modifications and additions further to the disclosed methods discussed herein may be made without departing from the spirit and scope of the invention.

### MPEP 706/707 Statement

If for any reason this application is not believed by the Examiner to be in full condition for allowance, applicant respectfully requests constructive assistance and suggestions of the Examiner, pursuant to M.P.E.P. 706.03 (d) and 707.07(j) in order that the applicants can place this application in allowable condition as soon as possible.

We claim:

1. A nasal mask cushion sealing apparatus for use in positive airway pressure therapy wherein;
The cushion sealing means includes protrusions which act to provide a primary sealing zone at the surface of the nares or within the nares cavity;
The cushion sealing means provides a secondary seal over the nasal bridge and around the periphery of the nose;

2. The apparatus in claim 1 wherein the seal around the perimeter of the nose in the region of the nasal bridge may be further enhanced by providing a sculptured portion to facilitate closer fit of tissues of the nasal bridge with the mask cushion.

3. The apparatus in claim 2 wherein the seal around the perimeter of the nose may be further enhanced by providing an additional flexible membrane which acts to form a stretch fit seal at the bridge of the nose or other areas of significant topographical variation and suited to such stretching means.

4. The apparatus in claim 2 or claim 3 wherein the cushion seal may be molded as a heavy walled construction from a low durometer elastomer

5. The apparatus in claim 4 wherein the primary nares sealing protrusions may be fitted independently after manufacture

6. The apparatus in claim 4 or 5 wherein the primary nares sealing protrusions may be constructed in a range of depths, to suit a variety of facial features and preferences, and to enable insertion into the nares or to be seated on the surface of the nares or partly within the nares.

7. The apparatus in claims 2 or 3 wherein the cushion seal may be molded as a thin walled profile from an elastomer of variable durometer sufficient to maintain its form.

8. The apparatus in claim 7 wherein the molded nares seals are formed as a simple protruding element and manufactured with a range of degree of protrusions

9. The apparatus in claim 7 wherein the molded nares seals are formed as a bellows.
